(19) United States
(12) Patent Application Publication
(10) Pub. No.: US 2011/0213258 A1
(43) Pub. Date: Sep. 1, 2011

(54) STRAP BASED RELIABLE HEART RATE OR ELECTRO CARDIOGRAM MONITOR WITH WIRELESS DATA TRANSMISSION

(76) Inventor: RAVINDRA WIJESIRIWARDANA, Bentonville, AR (US)

(21) Appl. No.: 13/037,254

(22) Filed: Feb. 28, 2011

Related U.S. Application Data
(60) Provisional application No. 61/308,964, filed on Feb. 28, 2010.

(51) Int. Cl. A61B 5/0402; A61B 5/04

(52) U.S. Cl. 600/509; 600/372

(57) ABSTRACT

This innovation describes a heart rate and an electro cardiogram monitoring system capable of operating under sweaty, high motion conditions and under water environments. The system is tailor made for ones physical parameters.

### CROSS-REFERENCE TO RELATED APPLICATIONS

This application claims the benefit of the U.S. provisional patent application 61/308,964 filed 2010 Feb. 28 by the present inventor. This application claims the benefit of the USA non-provisional patent application 20090292193 filed 2009 Mar. 11 by the present inventor. This application claims the benefit of provisional patent application Ser. No. 61/035,852, filed 2008 Mar. 12 by the present inventor. This application claims the benefit of provisional patent application Ser. No. 61/302,320, filed 2010 Feb. 8 by the present inventor. This application claims the benefits of the U.S. non provisional patent application Ser. No. 13/005,546 filed on 13 Jan. 2011

### FEDERALLY SPONSORED RESEARCH

Not Applicable

### SEQUENCE LISTING OR PROGRAM

Not Applicable

### BACKGROUND

1. Field

This application relates to bio-potential electrodes and sensors based wearable physiological information monitoring straps.

2. Prior Art

This innovation discusses a wireless heart rate or electro cardiogram (ECG) monitoring strap that is capable of providing heart rate or ECG information reliably.

Present day heart rate monitoring straps have two electrodes touching the skin, one on the left hand side and the other on the right hand side from the centre vertical chest plane of the wearer. After 10 people experimental study it was found that the locations of the electrodes are critical in minimizing the motion artifacts during the wearer undergoes activities.

An experiment was set up to analyze the motion artifacts against the locations of the electrodes on the strap. A heart rate monitoring strap with same electrodes and same physical parameters with electrodes on the left and right side of the chest of the vertical center plane (sagittal plane)of the chest against a heart rate monitor having electrodes in the heart side of the wearer are tested. The test results showed (FIG. 6A) that the signal to noise ratio of the strap containing the electrical activity pickup electrodes on the heart side of the sagittal plane on the chest of wearer are higher than the other heart rate monitor under the test. The test was carried out with 10 people with different age levels and the average results are shown in FIG. 6A.

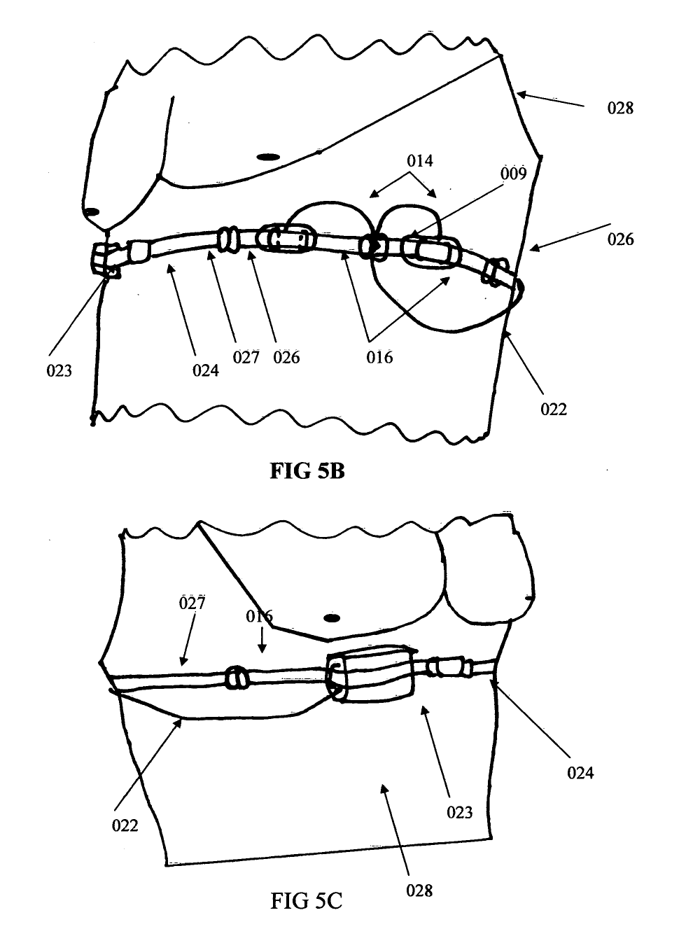

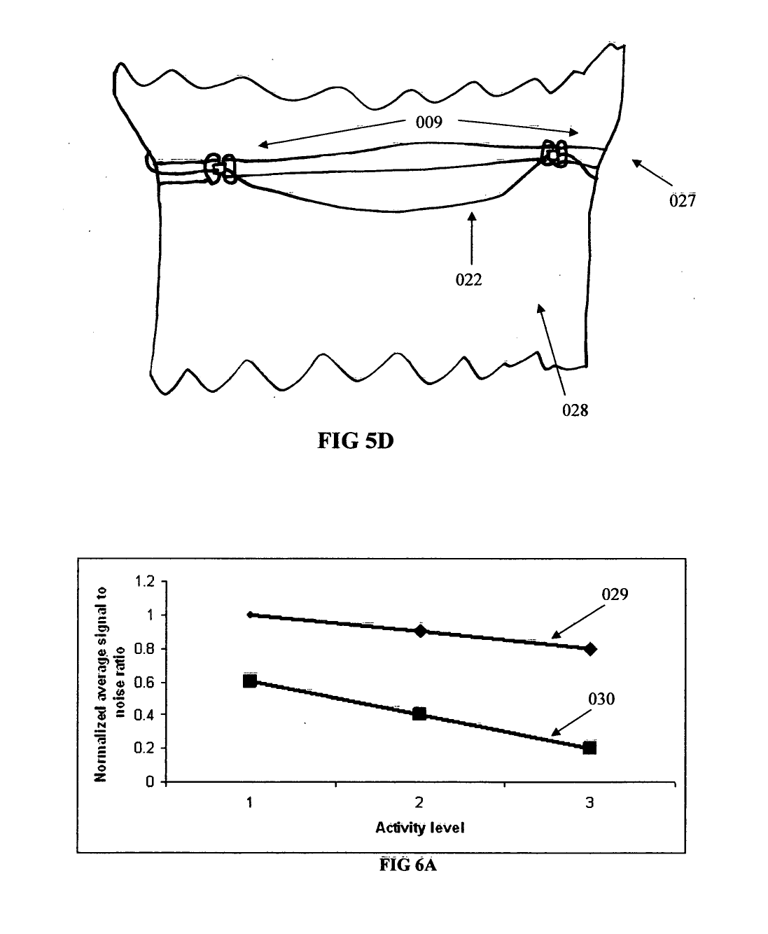

The present innovation is about this reliable heart rate monitor having electrode on the heart side of the strap (FIG. 5A and FIG. 5B).

### FIGURES

### Description

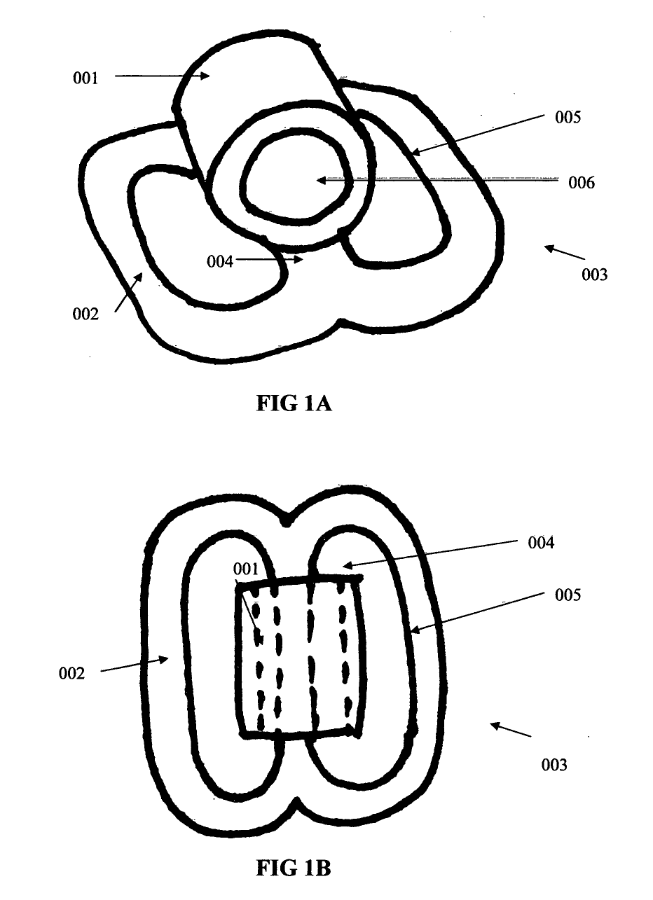

FIG. 1A: isometric view of a buckle with two loop holes and a third hole on the middle bar of the base parallel to the longer axis of the two holes on the base

FIG. 1B top view of the buckle 003

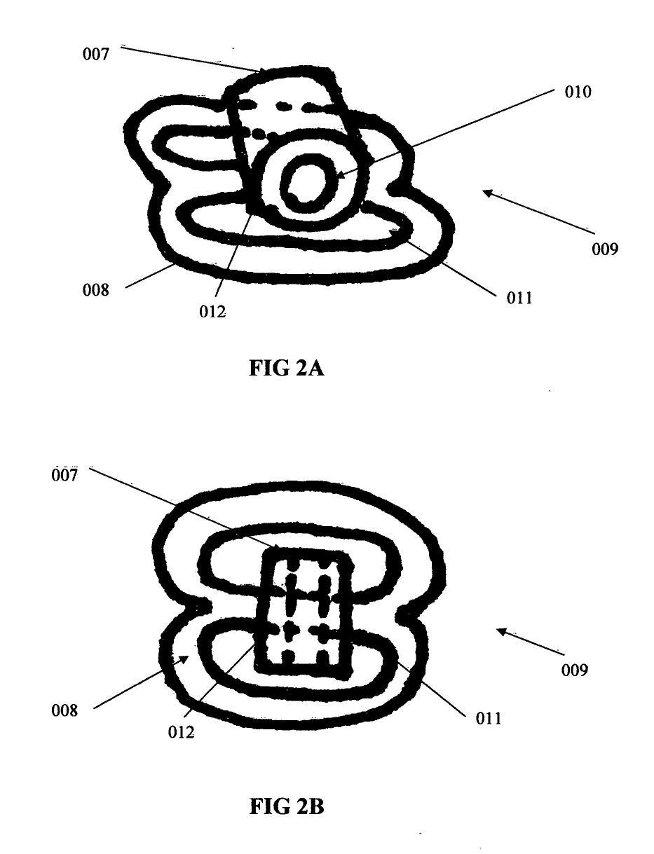

FIG. 2A Isometric view of a buckle with two loop holes on the base and a third hole on the middle bar of the base the third hole axis is not parallel to the longer axis of a hole on the base

FIG. 2B Top view of the buckle 009

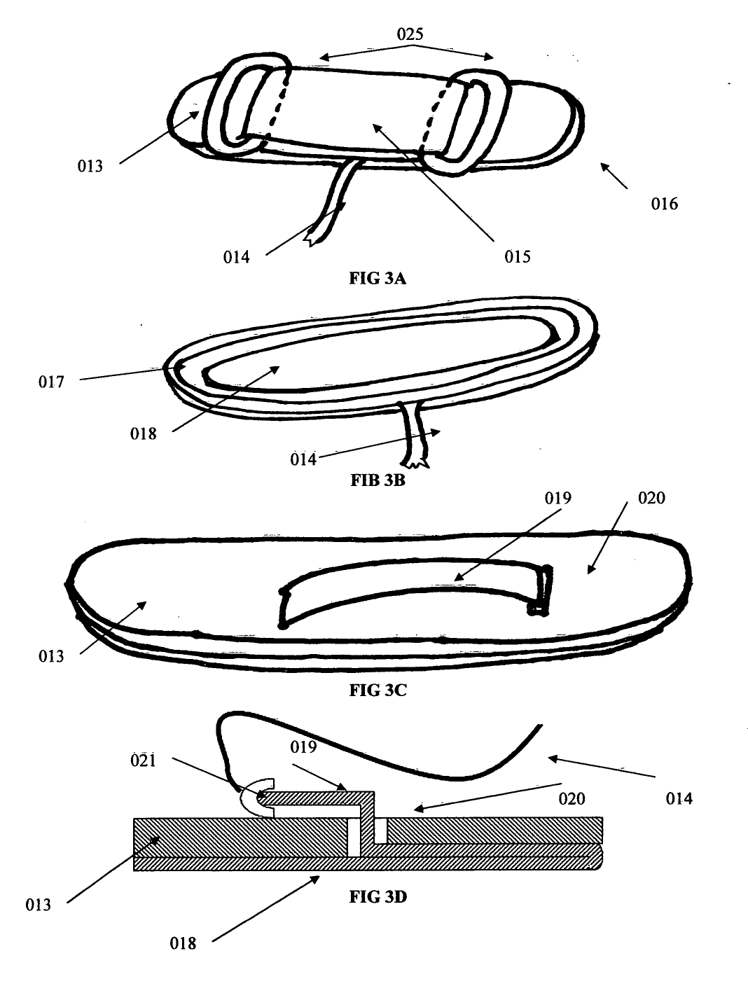

FIG. 3A A sensor base having two loop holes showing the loop side

FIG. 3B A sensor base having two loop holes showing the sensor element side

FIG. 3C the sensor Base layer show the electrical conduction pathways of the sensor element from the sensor element side comes to the other side through a hole in the base layer

FIG. 3D shows the cross sectional view of sensor base

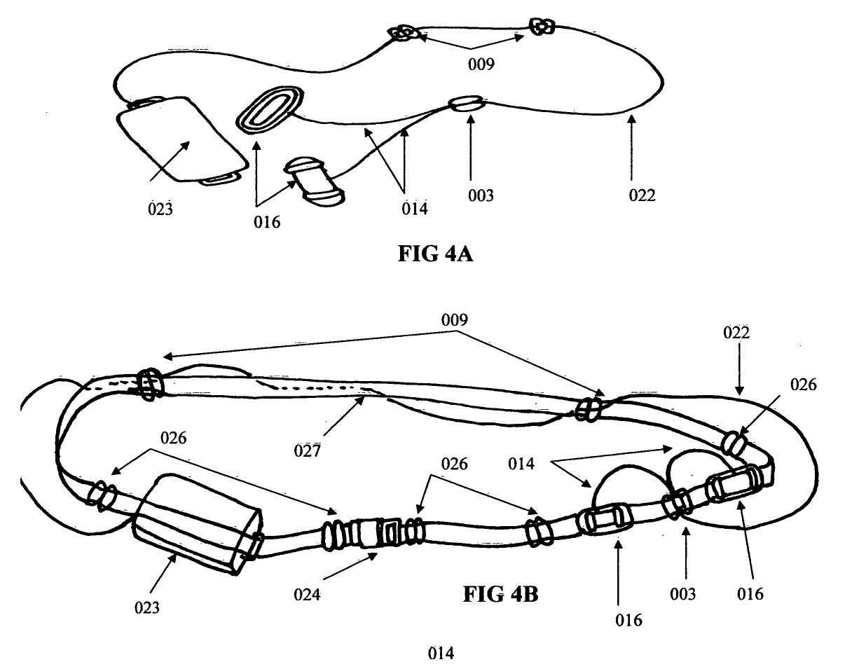

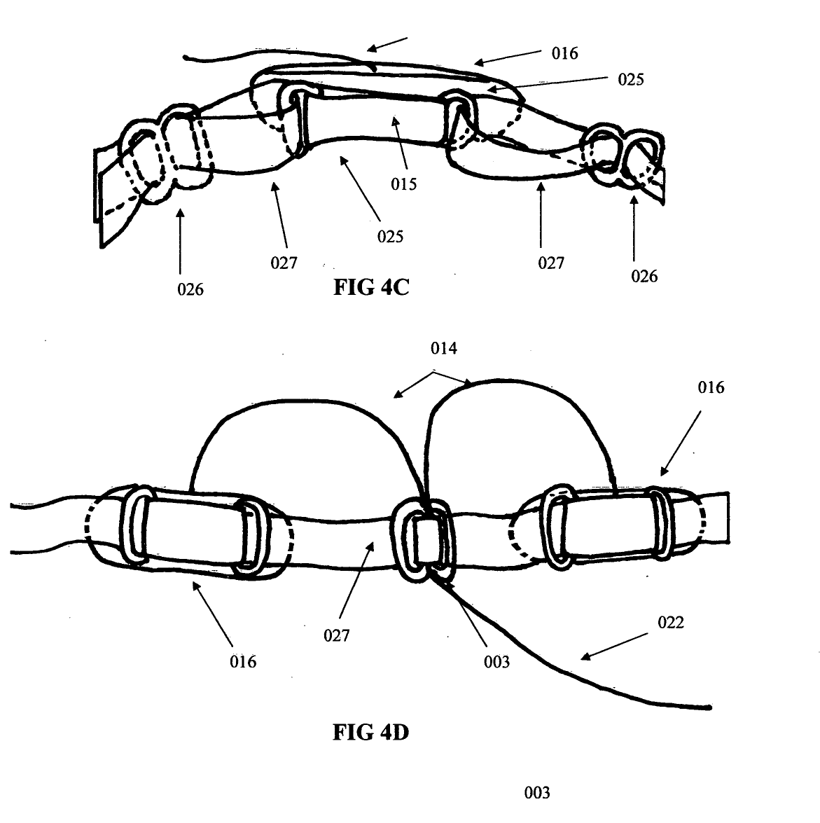

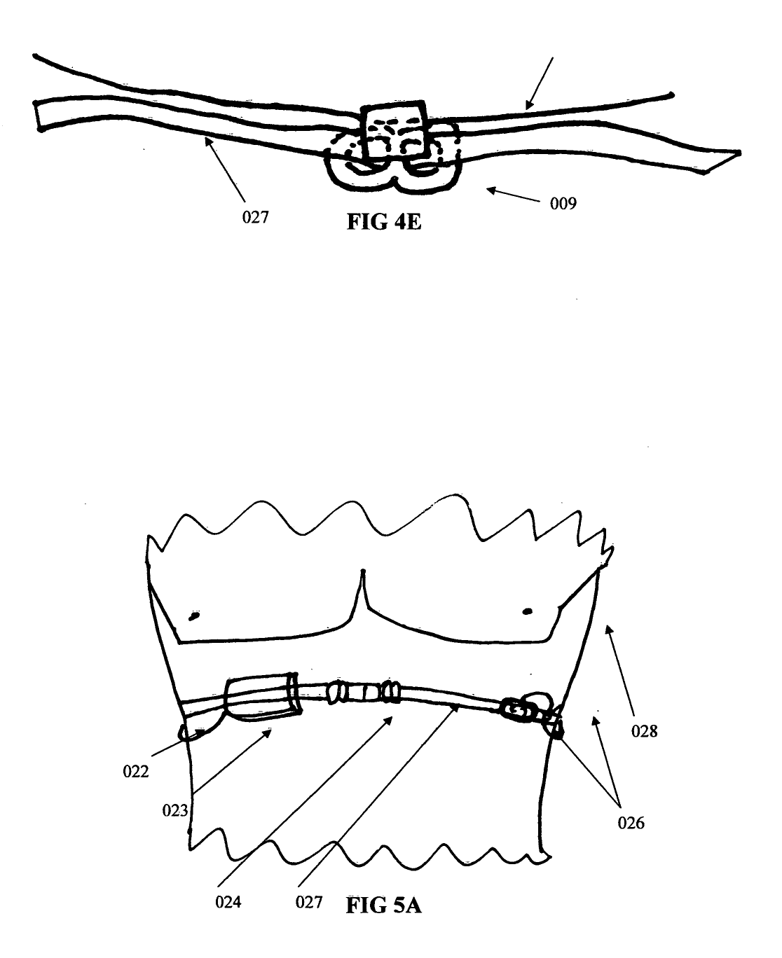

FIG. 4A A wireless physiological information system with sensor elements without the strap.

FIG. 4B shows the strap base ECG and Heart rate monitoring system with the electrode sensor bases are on the heart side of the wearer and the casing containing the electronics are placed on the right side of the wearer.

FIG. 4C shows the strap and the sensor base connection

FIG. 4D shows how the electrode sensor bases are connected to the moveable element or buckle such as 003 and the electrical connections between 014s and 0222 conductive pathways are inside the hole of the moveable segment.

FIG. 4E Shows the conductive pathways 022 goes through the hole of the moveable element or the buckle 009.

FIG. 5A Shows front view of the electrode strap worn by a person chest where the electrodes are on the heart side of the torso.

FIG. 5B shows the heart side view of the heart electrical activity monitoring strap with the electrodes on the heart side.

FIG. 5C shows the right hand side view of the heart electrical activity monitoring strap with the casing side.

FIG. 5D Shows the back view of the person wearing the heart electrical activity monitoring strap with the conduction pathways are laid inside he movable elements 009.

FIG. 6A Shows the graph of the Signal to noise ratios of heart rate monitor with electrodes on either side of the chest of the wearer and the heart rate monitor that the electrodes are on the heart side of the wearer.

### REFERENCE NUMERALS

001—a hollow cylinder on the middle bar parallel to the longer axis of the hole on the base or the middle bar
002—base of the buckle having two loop holes
003—A buckle with two loop holes having a third hole on the middle bar parallel to the longer axis of the two holes on the base
004—middle bar of the base
005—loop holes of the base
006—hole of the hollow cylinder 001
007—a hollow cylinder on the middle bar not parallel to the longer axis of the hole on the base or the middle bar
008—base of the buckle having two loop holes
009—a buckle with two loop holes having a third hole on the middle bar not parallel to the longer axis of the two holes on the base or the middle bar
010—the hole of the cylinder 007
011—loop holes of the base
012—middle bar of the base
013—the sensor base layer
014—the electrical conductive pathways between the sensor elements and the electrical circuits.
015—the layer where the loops are connect to the base
016—A sensor base with loops on one side and sensor elements on the other side
017—ring around the sensor element
018—sensor element that touches the skin
019—conduction pathways from the sensor element to the other side through the hole 020
020—a hole in the sensor base
021—conductive crimp connector or clip connector that connects the conductive pathways 019 of the sensor element and the conductive pathways to the electrical circuits.
022—electrical conduction pathways that connects the 014s to the electrical circuitry in the casing 023
023—case that covers the electrical circuits.
024—strap connector buckle
025—loops of the sensor Base
026—moveable stopper elements or buckles.
027—elastic strap
028 body of a human wearing the heart electrical activity monitoring strap.
029—signal to noise ratio of the heart rate monitoring strap with electrodes are on the heart side of the strap.
030—signal to noise ratio of the heart rate monitoring strap with electrodes of the strap are on the either side from the central plane of the wearer of the strap

1. A buckle with at least three separated loop holes, where at least two holes are in the same plane or the base plane and at least one other hole on a section of the base that separates the holes on the base of the buckle, such that the holes on the base of the buckle and the holes on the sections that separates the base holes of the buckle are in two non parallel planes.

2. A buckle according to claim 1 where the loop holes in the base and at least one additional third hole on the middle bar of the base parallel to the longer axis of the two holes on the base or the middle bar of the base.

3. A buckle according to claim 1 where the loop holes in the base and at least one additional third hole on the middle bar of the base such that the third hole axis is not parallel to the longer axis of a hole on the base or not parallel to the middle bar of the base.

4. A sensor base with at least one loop hole on one side and the sensor elements on the other parallel side so that it can be connect to a strap via the strap is passing through the loop hole/s and sensor element touches the wearers skin.

5. A sensor base according to claim 3 where there are at least two loop holes such that, each loop hole is connected to the part of the strap separately such that the sensor base cannot be moved along the strap.

6. A sensor base according to claim 4 or claim 5 where the strap parts are secured by using double loop buckles at each loop hole of the base.

7. A sensor base according to claim 4 or claim 5 or claim 6 where the electrically conductive pathways that connects the sensor elements to the external processing circuitry is passes through a holes or holes in the sensor base.

8. A sensor base according to claim 4 or claim 5 or claim 6 or claim 7 where there is a ring around the sensor elements.

9. A sensor base according to claim 4 or claim 5 or claim 6 or claim 7 or claim 8 where the sensor element is a bio-potential electrode.

10. A bio-potential electrode according claim 9 where the electrode is made of electro conductive cloth or electro conductive ink or hydro gel or wet electrode or dry electrode or any combination of.

11. A bio-potential electrode according to claim 10 having an electro-conductive fabric sensor element where part of the conductive fabric is goes through a hole in the sensor base to the other side of the sensor base and connects to a electrically conductive crimp connector or an electrically conductive clip and this crimp connector or the clip is connected to the conduction pathways that connects the electrode to the signal processing circuitry.

12. A sensor base according to claim 4, 5, 6, 7, 8, 9, 10 or 11 constructed with elastomeric materials, non elastomeric materials, foam or plastic or any combination of.

13. A sensor base according to claim 4, 5, 6, 7, 8, 9, 10, 11 or 12 constructed with layers.

14. A wireless hear rate or ECG monitoring system with at least two electrodes bases according to claim 4, 5, 6, 7, 8, 9, 10, 11, 12 or 13 where
a. the electrodes are connected to the signal processing circuitry via insulted wires;
b. the signal processing unit is covered with a plastic or ceramic covering having loop holes or no loop holes;
c. the insulated wires are inserted through at least one buckle according to claim 1 or claim 2 or claim 3.

15. A devise according to claim 14 where an elastic strap/s or parts of the strap are inserted via the holes of the casing, buckles, and the holes of the electrodes bases such that the Device A according to the claim 14 can be separated from the strap.

16. A device according claim 15 where the strap is inserted into moveable against the strap stopper elements such that these elements are placed on the specific locations along the strap to stop the slipping of the strap against the wearer.

17. A device according to claim 16 where the stopper elements are non stretchable having at least one hole.

18. A device according to claim 16 where the stopper element is a buckle or a buckle according to claim 1 or claim 2 or claim 3.

19. A device according to claim 14 or claim 15 or claim 16 or claim 17 or claim 18 where at least two electrode sensor bases that use ECG or heart rate detection is on the heart side or left hand side from the vertical center plane of the wearer and the signal processing and wireless transmitting device is on the other side of this plane.

20. A device according to claim 14 or claim 19 where the conductive pathways form the sensor bases are connected to a single electrical cable or wire having multiple strands at a buckle according to claim 1 or claim 2 or claim 3 and this buckle is connected to a strap according to claim 15.

21. A device according to claim 17 or claim 18 or claim 19 or claim 20 where the stopper element is made out of plastic, rubber or fabric or leather or foam or any combination of.

22. A device according claim 19, 20 or 21 where the wires charring the signal from the sensor bases are embedded in or on the strap.

23. A device according to claim 19, 20, 21 or 22 where the sensor bases are embedded in or on the strap.

24. A device according to claim 19, 20, 21, 22 or 23 capable of transmitting information wirelessly.

25. A device according to claim 24 where wireless communication is via the existing common wireless systems and protocols.

26. A device according to claim 24 or 25 having a wearable or non wearable display unit of the information.

27. A device according to claim 24, 25 or 26 where the strap is made with elastomeric or non elastomeric materials, silicone, acrylics, fabrics or any combination of.
